(19) United States
(12) Patent Application Publication
(10) Pub. No.: US 2004/0197404 A1
(43) Pub. Date: Oct. 7, 2004

(54) Extended release oral dosage form

(76) Inventors: Karin Ellstrom, Sodertalje (SE); Ulf Kjellberg, Sodertalje (SE); Hakan Nyqvist, Sodertalje (SE); Anders Ringberg, Sodertalje (SE); Annika Schweighofer, Sodertalje (SE)

Correspondence Address: WHITE & CASE LLP, PATENT DEPARTMENT, 1155 AVENUE OF THE AMERICAS, NEW YORK, NY, 10036, US

(21) Appl. No.: 10/488,125

(22) PCT Filed: Aug. 28, 2002
(86) PCT No.: PCT/SE02/01541

(30) Foreign Application Priority Data
Aug. 29, 2001 (SE) 0102887-7

(51) Int. Cl. A61K 9/22

(52) U.S. Cl. 424/468

(57) ABSTRACT

The present invention relates to a new extended release oral dosage form of a good soluble pharmaceutically active substance. More particularly, the invention relates to an extended release oral dosage form that provides a defined blood concentration profile having no rapid initial rise in blood plasma concentration of the good soluble active substance when administered at low dose. The invention further relates to processes for preparing said dosage form, the use of said dosage form and a method of prevention and/or treatment of CNS disorders and related medical disturbances using said dosage form.

### FIELD OF THE INVENTION

The present invention relates to a new extended release oral dosage form of a good soluble pharmaceutically active substance. More particularly, the invention relates to an extended release oral dosage form that provides a defined blood concentration profile having no rapid initial rise in blood plasma concentration of good soluble active substance when administered at low dose. The invention further relates to processes for preparing said dosage form, the use of said dosage form for the manufacture of a medicament and a method of prevention and/or treatment of CNS disorders and related medical disturusing said dosage form.

### BACKGROUND OF THE INVENTION

The development of new pharmaceutical active substances is often hampered or even blocked due to side effects of these new active substances. Some of the side effects may be overcome by developing suitable pharmaceutical formulations. This is for example true for substances that have a blood plasma concentration profile that starts with a rapid initial rise in blood plasma concentration which creates an early sharp and high peak plasma concentration. This early and high peak plasma concentration of the active substance can cause severe side effects. This problem can be overcome by altering the blood plasma concentration profile so that a more gradual absorbtion rate is obtained. Also, a short half-life of active substances can lead to low and insufficient concentrations in the blood plasma of the active substances at the end of a dosage interval. Increasing the dose can overcome this low and insufficient blood concentration. However, it is well known that administration of large doses of an active substance increases the risk for side effects. This is especially true for active substances that have their site of action in the brain. Administration of large doses of an anti-depressant drug can, for example, cause side effects such as dizziness, nausea, vomiting, etceteras. In the prevention and/or treatment of CNS disorders and related medical disturbances, the use of good soluble active substances having a short half-life and/or having a rapid initial rise in blood plasma concentration may therefore be limited.

Extended release dosage forms can be used to overcome these problems.

An extended release oral dosage form makes it possible to deliver an active substance to the blood in a controlled way such that the initial sharp rise in blood plasma concentration of the active substance is avoided. Another advantage of extended release oral dosage forms is the possibility to administer the prescribed daily dose of the active substance in the form of one unit dose while maintaining the desired therapeutic response over a period of up to 24 hours. In this way, the administration will be more user-friendly. Furthermore, the risk for therapeutic inefficiency due to bad compliance to frequent dosing and the lack of dosing during the night can be minimised. Extended release oral dosage forms further have the potential for improving treatment of e.g. chronic diseases. Besides, both systemic and local side effects, e.g. gastrointestinal irritation due to high local concentrations of the active substance, can be reduced.

A lot of research has been done with regard to the development of new extended release oral dosage forms. This has resulted in complicated formulations such as for instance entirely or partly coated multi-layer tablets containing the active substance in different amounts in the different layers and/or coating. In spite of all these developments, in general, extended release oral dosage forms still have some disadvantages. Development of an extended release oral dosage form whereby the time of release for a good soluble active substance can be varied is still a challenge. Furthermore, good soluble active substances with a short half-life are difficult to formulate in an extended release oral dosage form using a gel-forming polymer. Another problem is that the rate and/or extent of bioavailabilty are often influenced by the different physiological conditions of the gastrointestinal tract such as pH, enzyme activity and food intake. Especially food intake may cause problems with regard to the rate and/or extent of bioavailabilty. Another problem is related to dose dumping, i.e. a large amount of the dose is released in a short period of time.

### BRIEF DESCRIPTION OF THE INVENTION

It has now surprisingly been found that the pharmaceutical extended release oral dosage form of the present invention avoids the above-mentioned problems. The present invention provides for an extended release dosage form which is especially suitable for a good soluble active substance comprising a homogeneous mixture of the good soluble active substance, a gel-forming polymer and optionally other excipients, whereby the amount of the active substance is preferably low in the dosage form (<10% w/w). The dosage form may be coated or uncoated. The oral dosage form of the present invention provides for a defined release profile of the active substance in vivo over a predetermined period of up to 24 hours, whereby also the rapid initial rise in blood plasma concentration of the active substance is avoided. This means that there are less fluctuations of the blood plasma concentration over time and thus less risk for adverse effects due to such fluctuations as compared to an immediate release pharmaceutical dosage form. The prolonged and controlled plasma level of the active substance makes it possible to administer the active substance once or twice a day, thereby increasing patient compliance. The extended release oral dosage form according to the present invention provides a blood concentration profile of the active substance having a slower rise in peak plasma concentration compared to a conventional immediate release dosage form, thereby avoiding the adverse effects related to high peak plasma concentrations.

Active substances for use in the present invention are good soluble pharmaceutically active substances which for example may be used for in the prevention and/or treatment of disorders and related disturbances in the CNS. The present invention is especially suitable for substances having a short half-life and/or a rapid initial rise in blood plasma concentration. Of particular interest are 5-hydroxytryptamine receptor agonists, partial agonists or antagonists, preferably (R)-3-N,N-dicyclobutylamino-8-fluoro-3,4- dihydro-2H-1-benzopyran-5-carboxamide in the form of the free base or pharmaceutically acceptable salts and/or hydrates or solvates thereof. Particularly, the salt (R)-3-N,N-dicyclobutylamino-8-fluoro-3,4-dihydro-2H-1-benzopyran-S-carboxamide hydrogen tartrate. This tartrate comprises any of the optical forms (2R,3S), (2R,3R) and (2R,3S). Of these forms (R)-3-N,N-dicyclobutylamino-8-fluoro-3,4dihydro-2H-1-benzopyran-5-carboxamide hydrogen (2R,3R)-tartrate is preferred. The most preferred substance is (R)-3-N,N-dicyclobutylamino-8-fluoro-3,4-dihydro-2H-1-benzopyran-5-carboxamide hydrogen (2R,3R)-tartrate monohydrate (J. of Pharm. and Exp. Ther. (1997) Vol 283 No. 1, pp 216-225), which has a water solubility of 70 mg/ml at 25° C. and a plasma elimination half-life, t_{1/2} of 1.5 hours.

The blood plasma concentration 24 hours after administration (C₂₄) of an active substance, given in the extended release oral dosage form of the present invention, should be at least 25% of the maximum blood plasma concentration (Cₘₐₓ), preferably at least 40%, when the dose is administered orally to healthy fasting volunteers, and at least 13% of the maximum blood plasma concentration (Cₘₐₓ) when the administration of an active substance is combined with food intake (high fat standardised breakfast). The values for Cₘₐₓ and C₂₄ are average values from at least 11 volunteers.

The extended release oral dosage form according to the present invention provides a blood plasma concentration profile of the active substance having a slower rise in blood plasma concentration compared to a conventional immediate release dosage form, thereby avoiding the adverse effects related to high peak plasma concentrations. The time to reach the maximum blood plasma concentration (tₘₐₓ) for an active substance, after being administered in the extended release oral dosage form of the present invention, should be at least five times as long as the tₘₐₓ obtained after oral administration of said substance in an aqueous solution. The values have been calculated as the average values obtained from at least 35 volunteers.

Other parameters that can be used to express the blood plasma concentration profile of an active substance provided for by the extended release oral dosage form of the present invention are the area under the curve (AUC_{inf}), the mean residence time (MRT_{inf}) and the relative bioavailability (Fᵣₑₗ). The MRT_{inf} of an active substance reflects the mean time a molecule resides in the body. The MRT_{inf} of the extended release oral dosage form of the present invention should be at least three times as long as the MRT_{inf} of the same substance when administered orally in an aqueous solution to fasting volunteers. The MRT_{inf} is preferably between 5 and 15 hours, most preferably between 10 and 14 hours.

An aqueous solution shall mean a water solution containing the active substance. The present invention relates to an extended release oral dosage form that provides a release profile of the active substance that is rather unaffected by food intake. The influence of food intake should be such that the ratio of AUC_{inf}, MRT_{inf} and Fᵣₑₗ between food intake and non-food intake is between 0.8 to 1.3, preferable between 0.8 and 1.1. Further, the present invention relates to an extended release oral dosage form, which provides therapeutic levels of the active substance in blood plasma for at least 24 hours, and which has an in vitro dissolution profile in a phosphate buffer, pH 6.8, using USP Paddle method at 50 rpm, such that about 30 to 45.% of the active substance is released after 5 hours, about 60 to 75% is released after 10 hours and about 85 to 100% is released after 24 hours. The dosage form of the invention shall contain at least one gel-forming polymer, which may be selected from the group of hydroxymethyl cellulose, hydroxypropyl cellulose, hydroxyethyl cellulose, hydroxypropyl methylcellulose, methyl cellulose, ethyl cellulose, polyvinylpyrrolidone, polyethylene glycols, polyethylene oxide and poloxamers. Preferably, the gel-forming polymer is hydroxypropyl methylcellulose (HPMC). The viscosity of the HPMC should be between 3000 and 21000 cP, preferably 7500 and 21000 cP, and most preferably 11250 and 21000 cP of a 2% (w/w) aqueous solution at 20° C. The substitution degree of methoxy groups of this cellulose should be from 19 to 30% by weight, preferably from 19 to 28% and most preferably from 19 to 24% by weight and the substitution degree of hydroxypropoxy groups should be from 4 to 12% by weight, most preferably 7 to 12% by weight.

This HPMC may be mixed with a low viscosity HPMC. The low viscosity cellulose should have a viscosity within 3.75 and 140 cP, preferably from 11.3 to 140 cP and most preferably from 37.5 to 70 cP of a 2% (w/w) aqueous solution at 20° C. The substitution degree of methoxy groups of this cellulose should be from 19 to 30% by weight, preferably from 19 to 28% and most preferably from 19 to 24% by weight. The substitution degree of hydroxypropoxy groups should be from 4 to 12% by weight, most preferably 7 to 12% by weight.

The ratio of active substance to gel-forming polymer in the extended release oral dosage form of the present invention may be from 1:10 to 1:70, preferably from 1:20 to 1:50.

Beside the gel-forming polymers, the dosage form may optionally comprise excipients, e.g. binders, release modifying agents, lubricants, flow condition agents and the like. Suitable binders are hydroxypropyl cellulose, microcrystalline cellulose, polyvinylpyrrolidone, gelatine, polyethylene glycol, glycerylbehenate, glycerylmonostearate, carnauba wax and the like. The preferred binder is microcrystalline cellulose and/or polyvinylpyrrolidone. The amount of binder in the composition is from 0 to 45% w/w. The ratio of active substance to binder may be from 1:1 to 1:10, preferably from 1:2 to 1:6.

Other excipients that may be used in the dosage form are lubricants, such as magnesium stearate, sodium stearyl fumarate, stearic acid, polyethylene glycol, talc and the like, flow condition agents, such as colloidal silicon dioxide, talc and the like. As further excipients for the modification of the release, either slower or faster, can be mentioned lactose, mannitol, sorbitol, calcium phosphate, aluminium silicate, paraffin, carboxypolymethylene, carboxyvinyl polymer, acrylic acid polymer, ethyl cellulose, polyethylene glycol and the like. Additionally, excipients such as taste agents and colouring agents may be used. The amounts of these excipients are 0 to 55% w/w.

The dosage form may be prepared by mixing the active substance, the gel-forming polymers and optionally other excipients such as binders, lubricants and the like in a suitable mixer, e.g. a Turbula mixer, followed by direct compression of said homogeneous mixture.

Alternatively, the dosage form may be prepared from a granulated powder. The homogeneous powder may be obtained by mixing the active substance, the gel-forming polymers and optionally excipients such as binders in a suitable mixer. Then the mixture may be granulated in a mixer. The granulation can be performed in water or another granulation liquid such as an alcohol, e.g. ethanol, methanol, isopropanol, a ketone, e.g. acetone or aqueous mixtures thereof. From an environmental point of view water is preferred. The combination of PVP and HPMC in the ratio of 1 to 3, as used in one of the embodiments of the present invention, makes it feasible to use water as the granulation liquid instead of an organic liquid. The resultant wet granulation may thereafter be dried in a drying cabinet or in a fluid bed dryer and milled through a screen. The granulation may also be performed at elevated temperatures by using meltable binders. The cooled granulation may be milled through a screen. The dry granulate powder mass is then mixed with the remaining excipients and compressed into a suitable dosage form. Thirdly, the dosage form may be prepared by first compressing the dry granulate powder mass into loose compacts. These loose compacts may be milled through a screen and finally mixed with other excipients such as binders, lubricants and flow condition agents. The dry, homogeneous powder mass may then be manufactured into a suitable dosage form, e.g. compressed into tablets in a tablet machine. Other suitable oral dosage forms are capsules, minitablets and the like.

In a further embodiment of the invention the dosage form may comprise a coating layer. This coating layer may optionally have an extended release function and could contain additives for the modification of the release of the pharmaceutically active substance. Suitable polymers that can be used in the coating layer are ethyl cellulose, hydroxypropyl cellulose, hydroxypropyl methylcellulose, polyethylene glycol, acrylic and methacrylic esters such as Eudragit RL and RS (Röhm Pharma). Ethyl acrylate and methyl methacrylate such as Eudragit NE (Röhm Pharma) may also be used in the coating layer. The coating layer may further comprise binders such as microcrystalline cellulose, hydroxypropyl cellulose and the like, plastizicers such as polyethylene glycol, acetyl tributyl citrate and the like and colour agents such as titanium dioxide, iron oxide and the like. Also, antiadhesion agents such as colloid silicon dioxide, talc and the like may be used in the coating layer. The coating layer may additionally comprise taste-masking agents. As coating fluid may be used water or alcohols such as ethanol, optionally containing antibacterial agents such as hydrogen peroxide. The coating layer may be applied by way of spray coating in a fluidised bed, pan-coating or another coating technique known to a person skilled in the art. The extended release dosage form of the present invention is preferably coated.

The composition from which the dosage form is prepared can be formulated to contain the active substance in different amounts, e.g. between 0.1 and 100 mg, preferably between 0.5 and 50 mg, more preferably between 1 and 25 mg, particular preferred between 2.5 and 10 mg, but is not limited to these intervals. Suitable daily doses of the active substance may vary within a wide range and will depend on various factors such as the relevant disorder or medical conditions, the age, weight and sex, and may be determined by a physician.

The extended release oral dosage form according to the invention may be used in the prevention and/or treatment of CNS disorders and related medical disturbances, urinary incontinence, vasospasm or growth control of tumours, particularly, for 5-hydroxytryptainine mediated disorders and medical disturbances. Further, the extended release oral dosage form could for example be used in the prevention and/or treatment of affective disorders, mood disorders e.g. depression, major depressive episodes, dysthymia, seasonal affective disorder, depressive phases of bipolar disorder, anxiety disorders e.g. obsessive compulsive disorder, panic disorder with/without agoraphobia, social phobia, specific phobia, generalised anxiety disorder, posttraumatic stress disorder, personality disorders e.g. disorders of impulse control, trichotellomania, sleep disorders, eating disorders e.g. obesity, anorexia, bulimia, pre-menstrual syndrome, sexual disturbances, alcoholism, tobacco abuse, autism, attention deficit, hyperactivity disorder, migraine, memory disorders e.g. age associated memory impairment, presenile and senile dementia such as Alzheimer's disease, pathological aggression, schizophrenia, endocrine disorders e.g. hyperprolactinaemia, stroke, dyskinesia, Parkinson's disease, thermoregulation, pain, hypertension, urinary incontinence such as overactive bladder, detrusor instability, neurogenic bladder, detrusor hyperreflexia, nocturnal enuresis, e.g. bed-wetting in children, urinary frequency, urinary urgency, urge incontinence, stress incontinence, mixed incontinence, unstable bladder secondary to prostatitis or interstitial cystitis, disturbances of the cardiovascular system and disturbances in the gastrointestinal system.

The present invention also relates to processes for the manufacture of the extended release oral dosage form characterized by,

Method A, comprising the steps:

Ai) mixing the active substance with the gel-forming polymers and optionally binders, lubricants, modifying agents and other excipients,

Aii) forming the obtained dry powder mixture into a suitable solid dosage form and

Aiii) coating the obtained dosage form,

or,

Method B, comprising the steps:

Bi) mixing the active substance with the gel-forming polymers and optionally binders and other excipients,

Bii) granulating said mixture,

Biii) optionally drying the obtained granulate,

Biv) mixing the granulate with other excipients,

Bv) forming the obtained dry powder mixture into a suitable solid dosage form and

Bvi) coating the obtained dosage form, or,

Method C, comprising the steps:

Ci) mixing the active substance with the gel-forming polymers and optionally binders and other excipients,

Cii) granulating said mixture,

Ciii) optionally drying the obtained granulate,

Civ) compressing the granulate powder mass into loose compacts,

Cv) milling the compacts and mixing them with other excipients

Cvi) forming the obtained dry powder mixture into a suitable solid dosage form and

Cvii) coating the obtained dosage form.

The term ‘extended release oral dosage form’ shall mean any oral dosage form, which continuously releases the active substance at rates, which are sufficient to provide periods of prolonged therapeutic action following each administration of a single dose of such a dosage form. Alternative naming is e.g. controlled, sustained and slow release. According to the Biopharmaceutical Classification System used by the FDA, the term ‘good soluble’ shall means, the maximum dose to be administered, should be able to dissolve in 250 ml of an aqueous solution in the pH range of 1 to 8. The aqueous solution is preferably water.

Abbreviations;

CNS Central Nervous System

Cₘₐₓ maximum blood plasma concentration (nmol/L)

C₂₄ blood plasma concentration after 24 hours (nmol/L)

Cₜ blood plasma concentration after t hours (nmol/L)

t time (h)

t_{1/2} plasma elimination half-life (h)

AUC_{inf} Area Under the plasma concentration versus time Curve (nmol·h/L), calculated from time 0 to infinity.

MRT_{inf} Mean Residence Time (h), calculated from time 0 to infinity.

Fᵣₑₗ relative bioavailability

n number of persons participating in clinical tests

ER Extended Release

HPMC Hydroxypropyl methylcellulose

HPC (IF) Hydroxypropylcellulose (molecular weight approx. 95,000, pharmaceutical grade)

PEG Polyethylene glycol

ATBC Acetyl tributylcitrate

PVP Polyvinylpyrrolidone

HPLC High Pressure Liquid Chromatography

USP United States Phairmacopea

### EXAMPLES

The invention will now be illustrated by the following non-limiting examples.

### Example 1

The following components, expressed as mg per tablet, were used; batch size 2850 tablets:

| | |
|---|---|
| Active substance | 5 |
| HPMC (15000 cP) | 250 |
| PVP | 25 |
| Water | 100 |
| Microcrystalline cellulose | 55 |
| Colloidal silicon dioxide | 1.6 |
| Sodium stearyl fumarate | 3.3 |
| COATING | |
| HPMC (6 cP) | 4.2 |
| PEG (6000) | 1.0 |
| Titanium dioxide | 1.2 |
| Water | 115 |

HPMC (15000 cP) has a viscosity within the range of 11250-21000 cP and a substitution degree of methoxy groups of 19-24%9 by weight and hydroxypropoxy groups of 7-12% by weight. The viscosity values are given for 2% (w/w) aqueous solutions at 20° C.

The active substance, (R)-3-N,N-dicyclobutylamino-8-fluoro-3,4-dihydro-2H-1-benzopyran-5-carboxamide hydrogen (2R,3R)-tartrate monohydrate, HPMC (15000 cP) and polyvinylpyrrolidone were sieved through a 0.5 mm screen (or 1.0 mm screen for HPMC and PVP) and were mixed in a Turbula mixer for 10 minutes at a speed of 33 rpm. The powder mixture was granulated with water in an intensive mixer during water addition and additionally mixed for 2 minutes. The wet granulation was dried in a drying cabinet at 45° C. for 10 hours. The granulation was milled through a screen of 1.25 mm in an oscillating mill at 147 rpm. The granulation was compacted into loose compacts in a tablet machine equipped with punches of Ø 11 mm. The compacts were milled through a screen of 4 mm and then through a screen of 1.25 mm. The milled granulation was mixed with microcrystalline cellulose and colloidal silicon dioxide (screened through 0.5 mm) in a Turbula mixer for 6 minutes at a speed of 33 rpm. Sodium stearyl fumarate was added through a 0.5 mm screen and the mixing was continued for further 2 minutes.

The final homogeneous powder mixture was compressed into tablets in a tablet machine equipped with normally curved punches of Ø 10 mm.

The tablets were spray-coated in a tablet coating machine using an aqueous coating suspension of HPMC (6 cP) and PEG6000 and high speed homogenised suspended titanium dioxide.

In order to test the release of the active substance, (R)-3-N,N-dicyclobutylamino-8-fluoro-3,4-dihydro-2H-1-benzopyran-5-carboxamide hydrogen (2R,3R)-tartrate monohydrate, from the tablet an in vitro dissolution of the tablet was accomplished by using the USP Paddle method at 50 rpm. (Dissolution test, USP 24 p.1941)

Medium: Phosphate buffer, pH=6.8, 500 ml, Temperature: 37° C.

The following results were obtained:

| Time (h) | Amount dissolved (%) |
|---|---|
| 0 | 0 |
| 1 | 16 |
| 2 | 25 |
| 5 | 44 |
| 10 | 63 |
| 15 | 76 |
| 20 | 85 |
| 24 | 90 |

### Example 2

The following components expressed as mg per tablet were used; batch size 5800 tablets:

| | |
|---|---|
| Active substance | 5 |
| HPMC (15000 cP) | 120 |
| PVP | 12 |
| Water | 50 |
| Microcrystalline cellulose | 27 |
| Colloidal silicon dioxide | 0.8 |
| Sodium stearyl fumarate | 0.9 |
| COATING | |
| Ethylcellulose (10 cP) | 3.9 |
| HPC LF | 0.5 |
| Titanium dioxide | 0.9 |
| Ethanol 95 vol % | 179 |

In Example 2 the tablets were compressed using normally curved punches of Ø 8 mm. Otherwise the tablets were produced in the same manner as in Example 1.

The in vitro release of the active substance, (R)-3-N,N-dicyclobutylamino-8-fluoro-3,4-dihydro-2H-1-benzopyran-5-carboxamide hydrogen (2R,3R)-tartrate monohydrate, from the dosage form of Example 2 was obtained using the same method as in Example 1.

The following results were obtained:

| Time (h) | Amount dissolved (%) |
|---|---|
| 0 | 0 |
| 1 | 4 |
| 2 | 13 |
| 5 | 37 |
| 10 | 65 |
| 15 | 83 |
| 20 | 92 |
| 24 | 96 |

### Example 3

The following components expressed as mg per tablet were used; batch size 5800 tablets:

| | |
|---|---|
| Active substance | 5 |
| HPMC (15000 cP) | 120 |
| PVP | 12 |
| Water | 50 |
| Microcrystalline cellulose | 27 |
| Colloidal silicon dioxide | 0.8 |
| Sodium stearyl fumarate | 0.9 |
| COATING | |
| Eudragit ® RS30D | 4.9 |
| Eudragit ® RL30D | 1.6 |
| ATBC | 2.1 |
| Colloidal silicon dioxide | 2.0 |
| Talc | 2.7 |
| Titanium dioxide | 0.5 |
| Water | 161 |

In Example 3 the tablets were finally compressed using normally curved punches of Ø 8 mm. The coating suspension was prepared by stirring Eudragit RS30D, Eudragit RL30D, ATBC and part of the water for 19 hours. Colloidal silicon dioxide, talc, titanium dioxide and part of the water were high-speed mixed separately and then poured into the 19 hours stirred suspension. The coating layered tablets were coalesced in a drying cabinet at 45° C. for 15 hours. Otherwise the tablets were produced in the same manner as in Example 1.

The in vitro release of the active substance, (R)-3-N,N-dicyclobutylamino-8-fluoro-3,4-dihydro-2H-1-benzopyran-5-carboxamide hydrogen (2R,3R)-tartrate monohydrate, from the dosage form of Example 3 was obtained using the same method as in Example 1. The following results were obtained:

| Time (h) | Amount dissolved (%) |
|---|---|
| 0 | 0 |
| 1 | 8 |
| 2 | 18 |
| 5 | 42 |
| 10 | 70 |
| 15 | 85 |
| 20 | 94 |
| 24 | 97 |

Bioavailability

A single dose, three-way crossover bioavailability study was performed in 36 healthy volunteers. Three different extended release formulations of (R)-3-N,N-dicyclobutylamino-8-fluoro-3,4-dihydro-2H-1-benzopyran-5-carboxamide hydrogen (2R,3R)-tartrate monohydrate were tested. 11 to 12 volunteers tested each formulation. The extended release formulations were given in the fasting state and together with a high fat breakfast. The extended release oral dosage forms given were manufactured according to Examples 1-3 of the present invention. An aqueous solution was given as a reference formulation. The subjects received either 5 mg (R)-3-N,N-dicyclobutylamino-8-fluoro-3,4-dihydro-2H-1-benzopyran-5-carboxamide hydrogen (2R,3R)-tartrate monohydrate in the dosage form according to example 1 to 3 or 2.5 mg (R)-3-N,N-dicyclobutylamino-8-fluoro-3,4-dihydro-2H-1-benzopyran-5-carboxamide hydrogen (2R,3R)-tartrate monohydrate in the aqueous solution after overnight fasting. Plasma samples were withdrawn prior to and up to 36 hours after drug administration. Determination of (R)-3-N,N-dicyclobutylamino-8-fluoro-3,4-dihydro-2H-1-benzopyran-5-carboxamide hydrogen (2R,3R)-tartrate monohydrate in the plasma was performed using HPLC. The area under the plasma concentration of (R)-3-N,N-dicyclobutylamino-8-fluoro-3,4-dihydro-2H-1-benzopyran-5-carboxamide hydrogen (2R,3R)-tartrate monohydrate versus time curve (AUC_{inf}), the maximum plasma concentration (Cₘₐₓ), the plasma concentration after 24 hours (C₂₄), the time to reach the maximum blood plasma concentration (tₘₐₓ), the mean residence time (MRT_{inf}) and the relative bioavailability (Fᵣₑₗ) were calculated. The results, presented as average values obtained from each group of 11 to 12 volunteers are presented in Table A to C below.

TABLE A
Pharmacokinetic data obtained after
administration of the extended release oral
dosage form of Example 1. n = 11

| | Aqueous | | | | |
|---|---|---|---|---|---|
| | solution | ER_{fasting} | ER/Aq. | ER_{food} | |
| | (2.5 mg) | (5 mg) | sol. | (5 mg) | |
| Parameter | Fasting | Fasting | Fasting | Food | Food/Fast |
| Cₘₐₓ (nmol/l) | 102.2 | 38.6 | | 47.9 | |
| C₂₄ (nmol/l) | | 10.1 | | 7.5 | |
| (C₂₄/Cₘₐₓ) × 100% | | 26.2 | | 15.7 | |
| (%) | | | | | |
| AUC_{inf} | 263.3 | 588.7 | | 603.1 | 1.14 |
| (nmol * h/l) | | | | | |
| MRT_{inf} (h) | 2.38 | 12.72 | 5.35 | 10.19 | 0.80 |
| Fᵣₑₗ | | 1.08 | | 1.18 | 1.09 |

TABLE B
Pharmacokinetic data obtained after administration
of the extended release oral
dosage form of Example 2. n = 12

| | Aqueous | | | | |
|---|---|---|---|---|---|
| | solution | ER_{fasting} | ER/Aq. | ER_{food} | |
| | (2.5 mg) | (5 mg) | sol. | (5 mg) | |
| Parameter | Fasting | Fasting | Fasting | Food | Food/Fast |
| Cₘₐₓ (nmol/l) | 110.5 | 34.0 | | 56.1 | |
| C₂₄ (nmol/l) | | 17.7 | | 7.7 | |
| (C₂₄/Cₘₐₓ) × 100% | | 52.1 | | 13.7 | |
| (%) | | | | | |
| AUC_{inf} | 275.1 | 589.7 | | 579.3 | 1.26 |
| (nmol * h/l) | | | | | |
| MRT_{inf} (h) | 2.39 | 13.46 | 3.05 | 11.75 | 0.87 |
| Fᵣₑₗ | | 1.07 | | 1.10 | 1.03 |

TABLE C
Pharmacokinetic data obtained after
administration of the extended release oral
dosage form of Example 3. n = 12

| | Aqueous | | | | |
|---|---|---|---|---|---|
| | solution | ER_{fasting} | ER/Aq. | ER_{food} | |
| | (2.5 mg) | (5 mg) | sol. | (5 mg) | |
| Parameter | Fasting | Fasting | Fasting | Food | Food/Fast |
| Cₘₐₓ (nmol/l) | 94.2 | 43.2 | | 48.4 | |
| C₂₄ (nmol/l) | | 18.4 | | 7.0 | |
| (C₂₄/Cₘₐₓ) × 100% | | 42.6 | | 14.5 | |
| (%) | | | | | |
| AUC_{inf} | 256.4 | 782.6 | | 640.4 | 0.97 |
| (nmol * h/l) | | | | | |
| MRT_{inf} (h) | 2.52 | 13.83 | 5.49 | 11.35 | 0.82 |
| Fᵣₑₗ | | 1.50 | | 1.24 | 0.83 |

The results show that the extended release oral dosage form according to the present invention provides for a defined plasma concentration of a good soluble active substance over a period of at least 24 hours. The blood plasma concentration after 24 hours (C₂₄) is at least 25% of the maximum blood plasma concentration (Cₘₐₓ) in the fasting state, preferably at least 40% and at least 13% of Cₘₐₓ when administered together with food. The results also show that the MRT_{inf} increases at least three times when the active substance is administered in the extended release oral dosage form of the present invention compared to when administered orally in an aqueous solution to fasting volunteers. Beside, the results show that the effect of food intake is minimized after administration of a good soluble active substance in the extended release oral dosage form according to the present invention. The blood plasma profile of the active substance is affected by food intake such that the ratio of AUC_{inf}, MRT_{inf} and Fᵣₑₗ between food and no food intake is between 0.8 to 1.3, preferably between 0.8 and 1.1.

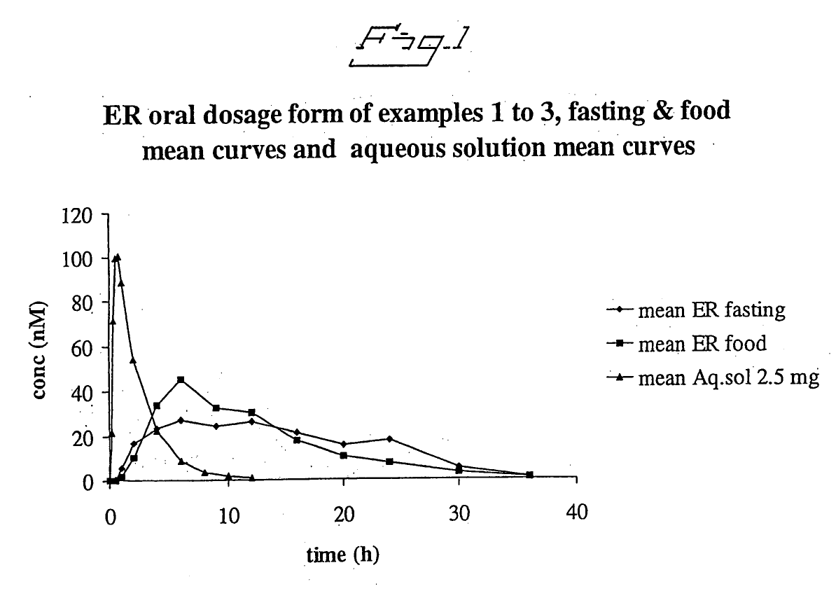

Further, it is shown that the extended release oral dosage form according to the present invention provides a blood plasma profile of the good soluble active substance with a prolonged time of the maximum peak concentration (tₘₐₓ). In FIG. 1 is shown that the time to reach maximum concentration (tₘₐₓ) is extended from 45 minutes to about 6 hours, after administration of a good soluble active substance in the extended release oral dosage form of the present invention.

There is no dose dumping when the active substance is administered in the extended release oral dosage form of the present invention.

FIG. 1. The average blood plasma concentrations obtained as average values obtained from 35 volunteers after administration of an active substance, (R)-3-N₃N-dicyclobutylamino-8-fluoro-3,4-dihydro-2H-1-benzopyran-5-carboxamide hydrogen (2R,3R)-tartrate monohydrate, in either an aqueous solution (2.5 mg) or in the extended release oral dosage forms according to the present invention with or without simultaneous food intake.

1. An extended release oral dosage form comprising a 5-hydroxytryptamine receptor agonist, partial agonist or antagonist as the active substance, in mixture with at least one gel-forming polymer and optionally one or more excipients.

2. The extended release oral dosage form according to claim 1, wherein the active substance is (R)-3-N,N-dicyclobutylamino-8-fluoro-3,4-dihydro-2H-1-benzopyran-5-carboxamide, in the form of a free base, hydrate, and/or solvate, or a pharmaceutically acceptable salt thereof.

3. The extended release oral dosage form according to claim 2, wherein the active substance is (R)-3-N,N-dicyclobutylamino-8-fluoro-3,4-dihydro-2H-1-benzopyran-5-carboxamide hydrogen (2R,3R)-tartrate.

4. The extended release oral dosage form according to claim 2, wherein the active substance is (R)-3-N,N-dicyclobutylamino-8-fluoro-3,4-dihydro-2H-1-benzopyran-5-carboxamide hydrogen (2R,3R)-tartrate monohydrate.

5. The extended release oral dosage from according to claim 1, wherein the gel-forming polymer is selected from the group consisting of hydroxymethyl cellulose, hydroxypropyl cellulose, hydroxyethyl cellulose, hydroxypropyl methylcellulose, methyl cellulose, ethyl cellulose, polyvinylpyrrolidone, polyethylene glycols, polyethylene oxides and poloxamers.

6. The extended release oral dosage form according to claim 5, wherein the gel-forming polymer is HPMC having a viscosity between 3000 and 21000 cP, a substitution degree of methoxy groups in the range of from 19 to 30% by weight, and a substitution degree of hydroxypropoxy groups in the range of from 4 to 12% by weight.

7. The extended release oral dosage form according to claim 1, wherein the binder is selected from the group consisting of microcrystalline cellulose, polyvinylpyrrolidone, glycerylbehenate and hydroxypropyl cellulose, and mixtures thereof.

8-10. (canceled)

11. The extended release oral dosage form according to claim 1, wherein the ratio of the active substance to gel-forming polymer is from 1:10 to 1:70.

12. The extended release oral dosage form according to claim 1, wherein the ratio of the active substance to binder is from 1:1 to 1:10.

13. The extended release oral dosage form according to claim 1, wherein the amount of the active substance in the dosage form is less than 10% w/w.

14. The extended release oral dosage form according to claim 1, wherein the dosage form is coated with a coating layer.

15. The extended release oral dosage form according to claim 14, wherein the coating layer comprises a polymer.

16. (canceled)

17. The extended release oral dosage form according to claim 15, wherein the coating layer further comprises one or more excipients selected from the group consisting of plasticizers, colouring agents, pigments, taste-masking agents, and antiadhesion agents.

18-34. (canceled)

35. The extended release oral dosage form according to claim 1, wherein the dosage form has a mean dissolution profile in vitro, in phosphate buffer at a pH of 6.8, using the USP Paddle method at 50 rpm, such that about 30 to 45% of the active ingredient is released after 5 hours, about 60 to 75% is released after 10 hours, and about 85 to 100% is released after 24 hours.

36. The extended release oral dosage form according to claim 1, wherein the dosage form upon administration in the fasting state provides a blood plasma concentration of the active substance after 24 hours that is at least 25% of the maximum blood plasma concentration (tₘₐₓ) and upon administration together with food provides a blood plasma concentration of the active substance after 24 hours that is at least 13% of the (tₘₐₓ).

37. The extended release oral dosage form according to claim 1, wherein the time to reach the maximum blood plasma concentration (tₘₐₓ) of the active substance is at least five times as long as the tₘₐₓ obtained when the substance is administered orally in an aqueous solution.

38. The extended release oral dosage form according to claim 1, wherein the dosage form upon administration in the fasting state provides a blood plasma concentration of the active substance after 24 hours that is at least 40% of the maximum blood plasma concentration (tₘₐₓ).

39. The extended release oral dosage form according to claim 1, wherein the dosage form upon administration provides a mean residence time (MRT_{inf}) of the active substance that is at least three times as long as the MRT^{inf} obtained when the active substance is administered orally in an aqueous solution under fasting conditions.

40. The extended release oral dosage form according to claim 1, wherein the mean residence time (MRT_{inf}) of the active substance is between 5 and 15 hours.

41. The extended release oral dosage form according to claim 1, wherein the dosage form comprises an amount of 1 to 25 mg of the active substance, and wherein upon administration, the dosage form gives a blood plasma profile of the active substance that is affected by food intake such that the ratio between administration together with food and administration on an empty stomach for each of AUC^{inf}, MRT^{inf} and Fᵣₑₗ, is between 0.8 to 1.3.

42. The extended release oral dosage form according to claim 1, wherein the excipients are selected from the group consisting of binders, lubricants, release modifying agents, flow condition agents, and pharmaceutically acceptable excipients.

43. The extended release oral dosage form according to claim 42, wherein the lubricant is selected from the group consisting of magnesium stearate powder, sodium stearyl fumarate, stearic acid, polyethylene glycol, and talc.

44. The extended release oral dosage form according to claim 42, wherein the flow condition agent is colloid silicon dioxide.

45. The extended release oral dosage form according to claim 42, wherein the release modifying agent is selected from the group consisting of lactose, mannitol, sorbitol, calcium phosphate, aluminium silicate, paraffin, carboxypolymethylene, carboxyvinyl polymer, acrylic acid polymer, ethyl cellulose, and polyethylene glycol.

46. The extended release oral dosage form according to claim 6, wherein the HPMC has a viscosity between 7500 and 21000 cP.

47. The extended release oral dosage form according to claim 6, wherein the HPMC has a viscosity between 11250 and 21000 cP.

48. The extended release oral dosage form according to claim 6, wherein the HPMC has a substitution degree of methoxy groups in the range of from 19 to 28%.

49. The extended release oral dosage form according to claim 6, wherein the HPMC has a substitution degree of methoxy groups in the range of from 19 to 24%.

50. The extended release oral dosage form according to claim 6, wherein the HPMC has a substitution degree of hydroxypropoxy groups in the range of from 7 to 12% by weight.

51. The extended release oral dosage form according to claim 11, wherein the ratio of active substance to gel-forming polymer is in the range of from 1:20 to 1:50.

52. The extended release oral dosage form according to claim 12, wherein the ratio of active substance to binder is in the range of from 1:2 to 1:6.

53. The extended release oral dosage form according to claim 15, wherein the polymer is selected from the group consisting of ethyl cellulose, hydroxypropyl cellulose, polyethylene glycol, low viscosity HPMC, acrylic and methacrylic esters, ethyl acrylate, methyl methacrylate, and mixtures thereof.

54. The extended release oral dosage form according to claim 53, wherein the coating layer comprises a mixture of hydroxypropyl cellulose and low viscosity ethyl cellulose.

55. The extended release oral dosage form according to claim 40, wherein the mean residence time (MRT_{inf}) is between 10 and 14 hours.

56. The extended release oral dosage form according to claim 41, wherein upon administration, the dosage form gives a blood plasma profile of the active substance that is affected by food intake such that the ratio between administration together with food and administration on an empty stomach for each of AUC_{inf}, MRT_{inf} and Fᵣₑₗ, is between 0.8 to 1.1.

57. A process for the manufacture of an extended release dosage form according to any one of claims 1-7, 11-15, 17, or 35-56, the process selected from the group consisting of:
method A, comprising the steps of:
Ai) mixing the active substance with at least one gel-forming polymer and optionally one or more pharmaceutically acceptable excipients,
Aii) forming the obtained dry powder mixture into a solid dosage form, and
Aiii) coating the obtained dosage form;
method B, comprising the steps of:
Bi) mixing the active substance with at least one gel-forming polymer and optionally one or more pharmaceutically acceptable excipients,
Bii) granulating the mixture,
Biii) optionally drying the obtained granulate,
Biv) mixing the granulate with one or more pharmaceutically excipients,
Bv) forming the obtained dry powder mixture into a solid dosage form and
Bvi) coating the obtained dosage form; and
method C, comprising the steps of:
Ci) mixing the active substance with at least one gel-forming polymer and optionally one or more pharmaceutically acceptable excipients,
Cii) granulating the mixture,
Ciii) optionally drying the obtained granulate powder mass,
Civ) compressing the granulate powder mass into loose compacts,
Cv) milling the compacts and mixing the milled compacts with one or more pharmaceutically acceptable excipients,
Cvi) forming the obtained dry powder mixture into a solid dosage form, and
Cvii) coating the obtained dosage form.

58. The process according to claim 57, wherein the granulation in step Bii and Cii is performed in water.

59. A method for the prophylaxis and/or treatment of a medical condition selected from the group consisting of disorders and related medical disturbances in the central nervous system (CNS), urinary incontinence, vasospasm, and growth control of tumours, the method comprising administering an effective amount of the extended release oral dosage form according to any one of claims 1-7, 11-15, 17, or 35-56 to a patient in need thereof.

60. The method according to claim 59, wherein the medical condition is a 5-hydroxytryptamine mediated disorder or disturbance.

61. The method according to claim 59, wherein the medical condition is depression, anxiety, or a memory disorder.

62. The method according to claim 59, wherein the medical condition is a disturbance of the cardiovascular system or a disturbance of the gastrointestinal system.

63. The method according to claim 59, wherein the medical condition is over-active urine bladder.

64. The method according to claim 61, wherein the medical condition is Alzheimer's Disease.
